(19) United States
(12) Patent Application Publication
(10) Pub. No.: US 2011/0137393 A1
(43) Pub. Date: Jun. 9, 2011

(54) STIFFINER HAVING AN ENLARGED BOMBOUS DISTAL END REGION AND CORRESPONDING COCHLEAR IMPLANT STIMULATING ASSEMBLY

(76) Inventors: Nicholas C. Pawsey, North Ryde (AU); Peter R. Sibary, Sydney (AU)

(21) Appl. No.: 12/630,101

(22) Filed: Dec. 3, 2009

(51) Int. Cl. A61F 11/04; A61N 1/05

(52) U.S. Cl. 607/137

(57) ABSTRACT

A cochlear implant comprising an elongate implantable stimulating assembly configured to be implanted in a recipient's cochlea, the stimulating assembly having a carrier member with a lumen longitudinally extending therethrough; and an elongate stylet configured to be removably inserted into the lumen, the stylet having an elongate body region and a distal end region comprising a bombous tip having a cross-sectional diameter that is approximately the same as the diameter of the lumen.

### BACKGROUND

1. Field of the Invention

The present invention relates generally to cochlear implants, and more particularly, to a stiffener having a bombous distal end region and a corresponding stimulating assembly of a cochlear implant.

2. Related Art

There are a variety of medical devices which provide a therapeutic benefit to a patient, user or recipient (“recipient” herein). Of particular relevance are implantable leads, catheters and the like having an elongate structure commonly referred to as a carrier member with an integrated lumen and corresponding stiffener to facilitate control of the configuration, orientation and/or positioning of the carrier member in the recipient.

There are a variety of carrier members which may be temporarily or permanently implanted in a recipient to provide a therapeutic benefit. For example, carrier members may be used to deliver pharmaceuticals, deploy sensors, remove natural or man-made fluids or gases, retrieve tissue samples, deliver or position an imaging device, deploy a surgical instrument, etc. One common use of a carrier member is for implanting electrode contacts that are subsequently utilized to deliver electrical, optical or other stimulation signals to a target tissue. One specific example is the elongate stimulating assembly commonly employed in cochlear implants. The stimulating assembly includes a flexible carrier member on which an array of electrode contacts is disposed. The carrier member is configured for implantation in a recipient's cochlea to position the electrode contacts at predetermined locations in the cochlea. The carrier member has an integrated lumen which receives a corresponding elongate stiffener (commonly referred to as a stylet). As noted, the stylet is used by a surgeon to control the configuration, orientation and/or position of the stimulating assembly in the recipient's cochlea.

### SUMMARY

In one aspect of the present invention, a cochlear implant is disclosed, the cochlear implant comprising: an elongate implantable stimulating assembly configured to be implanted in a recipient's cochlea, the stimulating assembly having a carrier member with a lumen longitudinally extending therethrough; and an elongate stylet configured to be removably inserted into the lumen, the stylet having an elongate body region and a distal end region comprising a bombous tip having a cross-sectional diameter that is approximately the same as the diameter of the lumen.

In another aspect of the present invention, an elongate stylet for use with an elongate stimulating assembly of a cochlear implant is disclosed. The stimulating assembly comprising an implantable carrier member having a lumen extending therethrough and a plurality of electrode contacts disposed on the carrier member. The stylet comprises an elongate body region; and a distal end region comprising a bombous tip having a cross-sectional diameter which is approximately the same as the diameter of the lumen.

### BRIEF DESCRIPTION OF THE DRAWINGS

Embodiments of the present invention are described below with reference to the attached drawings, in which:

FIG. 1 is a perspective view of an implanted cochlear implant which has been implanted with a stylet, in accordance with embodiments of the present invention;

FIG. 2A is a perspective, partially cut-away view of a human cochlea;

FIG. 2B is a cross-sectional view of one turn of the canals of the cochlea illustrated in FIG. 2A;

FIG. 3A is a side view of an embodiment of the implantable component of the cochlear implant illustrated in FIG. 1;

FIG. 3B is a side view of an embodiment of the stimulating assembly illustrated in FIG. 3A in a straightened configuration due to the presence of a stylet in the lumen of the carrier member;

FIG. 3C is a side view of an embodiment of the stylet illustrated in FIG. 3B, the stylet having an enlarged bombous distal end;

FIG. 3D is a cross-sectional view of the stimulating assembly illustrated in FIG. 3B taken along section line 3D-3D of FIG. 3B;

FIG. 3E is a side view of an embodiment of the stimulating assembly illustrated in FIG. 3A in a curved configuration due to the absence of a stylet in the lumen of the carrier member;

FIG. 3F is a cross-sectional view of the stimulating assembly illustrated in FIG. 3E taken along section line 3F-3F of FIG. 3E;

FIG. 4A is a side view of the enlarged bombous distal end region of an embodiment of the stylet illustrated in FIG. 3C;

FIG. 4B is a side view of the enlarged bombous distal end region of another embodiment of the stylet illustrated in FIG. 3C;

FIG. 4C is a side view of the enlarged bombous distal end region of a further embodiment of the stylet illustrated in FIG. 3C;

FIG. 5A is a schematic view of an embodiment of a stylet of the present invention in which the enlarged bombous distal end region of the stylet has a cross-sectional diameter or width which is approximately the same as the diameter of the carrier member lumen;

FIG. 5B is a schematic views of an embodiment of a stylet of the present invention in which the enlarged bombous distal end region of the stylet has a cross-sectional diameter or width which is slightly smaller that the diameter of the carrier member lumen;

FIG. 5C is a schematic views of an embodiment of a stylet of the present invention in which the enlarged bombous distal end region of the stylet has a cross-sectional diameter or width which is slightly larger that the diameter of the carrier member lumen; and

FIG. 6 is a schematic view of an embodiment of a stylet of the present invention positioned within a carrier member lumen subsequent to an improper reinsertion of the stylet into the lumen.

### DETAILED DESCRIPTION

Aspects of the present invention are generally directed to an implantable lead, catheter or the like comprising an elongate carrier member having an integrated lumen and corresponding stiffening element or stylet (“stylet” herein) with a clavate, bulbiform or other enlarged distal end region having a bombous tip and a cross-sectional diameter that approximates the diameter of the lumen.

There are a variety of carrier members which may be temporarily or permanently implanted in a recipient to provide a therapeutic benefit. For example, stimulating medical devices often include a carrier member to position electrode contacts at a desired location in a recipient. One specific example is a cochlear implant which includes an elongate stimulating assembly that is implanted in a recipient's cochlea to deliver stimulation to the auditory nerve.

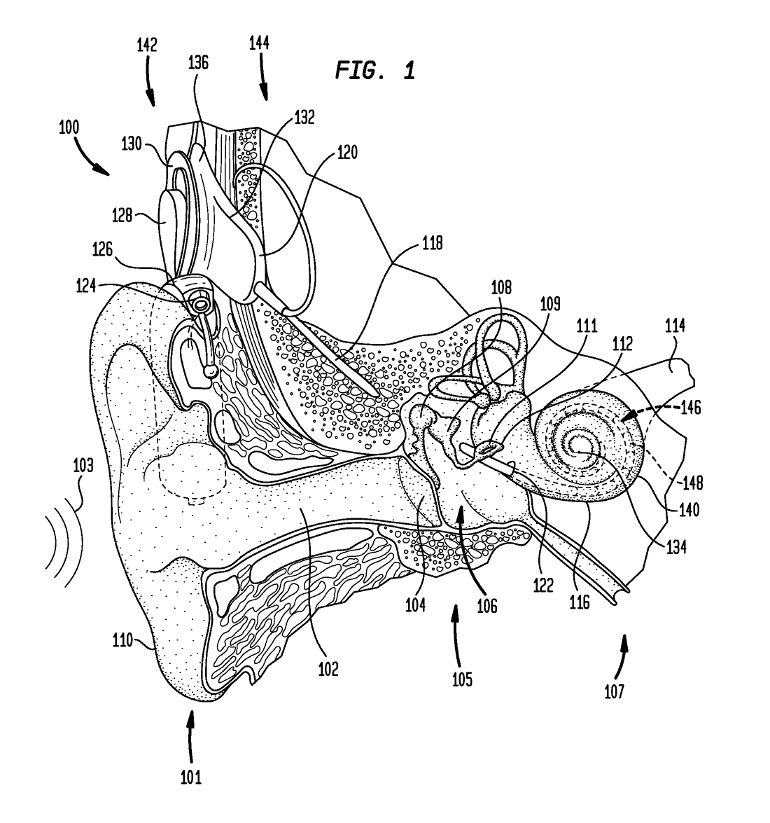

FIG. 1 is perspective view of an exemplary cochlear implant 100. In FIG. 1, cochlear implant 100 is shown implanted in a human cochlea. The relevant components of outer ear 101, middle ear 105 and inner ear 107 are described next below, followed by a description of cochlear implant 100.

In a fully functional ear, outer ear 101 comprises an auricle 110 and an ear canal 102. An acoustic pressure or sound wave 103 is collected by auricle 110 and channeled into and through ear canal 102. Disposed across the distal end of ear canal 102 is a tympanic membrane 104 which vibrates in response to sound wave 103. This vibration is coupled to oval window or fenestra ovalis 112 through three bones of middle ear 105, collectively referred to as the ossicles 106 and comprising the malleus 108, the incus 109 and the stapes 111. Bones 108, 109 and 111 of middle ear 105 serve to filter and amplify sound wave 103, causing oval window 112 to articulate, or vibrate. Such vibration sets up waves of fluid motion within cochlea 140. Such fluid motion, in turn, activates hair cells (not shown) that line the inside of cochlea 140. Activation of the hair cells causes appropriate nerve impulses to be generated. The nerve impulses are transferred through the spiral ganglion cells and auditory nerve 114 to the brain (also not shown), where they are perceived as sound.

Cochlear implant 100 comprises external component assembly 142 which is directly or indirectly attached to the body of the recipient, and an internal component assembly 144 which is implanted in the recipient. External assembly 142 typically comprises one or more audio pickup devices for detecting sound such as microphone 124, a sound processor 126, a power source (not shown), and an external transmitter unit 128. External transmitter unit 128 comprises an external coil 130 of a transcutaneous energy transfer arrangement. Sound processor 126 processes the electrical signals generated by microphone 124 that is positioned, in the depicted embodiment, by auricle 110 of the recipient. Sound processor 126 generates coded signals, referred to herein as a stimulation data signals, which are provided to external transmitter unit 128 via a cable (not shown).

Internal assembly 144 comprises an internal receiver unit 132, a stimulator unit 120, and an elongate electrode carrier 118. Internal receiver unit 132 comprises an internal coil 136 of the transcutaneous energy transfer arrangement. Internal receiver unit 132 and stimulator unit 120 are hermetically sealed within a biocompatible housing. The internal coil receives power and stimulation data from external coil 130, as noted above. Elongate electrode carrier 118 has a proximal end connected to stimulator unit 120 and extends from stimulator unit 120 to cochlea 140. Electrode carrier 118 is implanted into cochlea 104 via a cochleostomy 122.

Electrode carrier 118 comprises an electrode array 146 disposed at the distal end thereof. Electrode array 146 comprises a plurality of electrodes 148. Stimulation signals generated by stimulator unit 120 are applied by electrode contacts 148 to cochlea 140.

In some cochlear implants, external coil 130 transmits electrical signals (that is, power and stimulation data) to the internal coil via a radio frequency (RF) link. The internal coil is typically a wire antenna coil comprised of multiple turns of electrically insulated single-strand or multi-strand platinum or gold wire. The electrical insulation of the internal coil is provided by a flexible silicone molding (not shown). In use, implantable receiver unit 132 may be positioned in a recess of the temporal bone adjacent auricle 101 of the recipient.

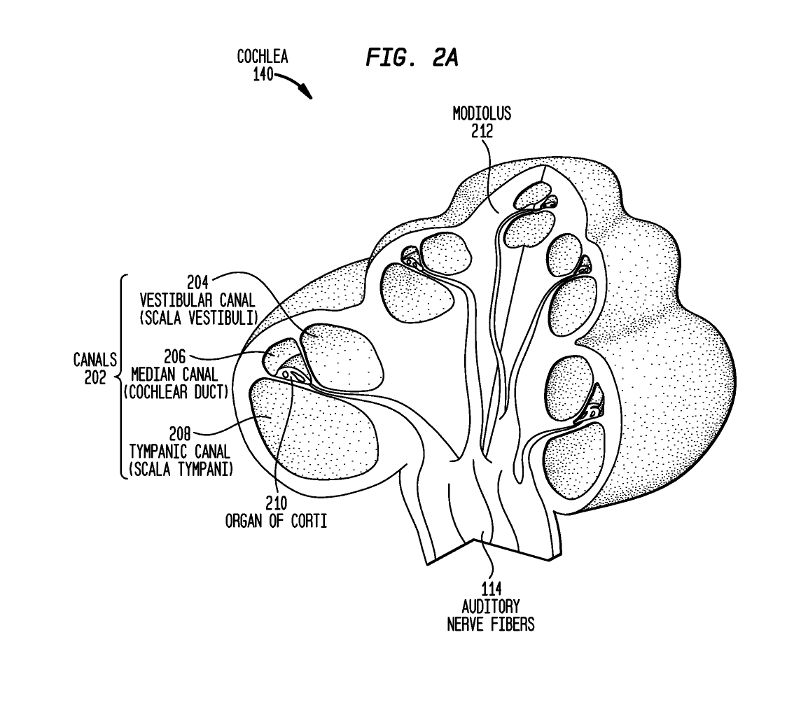

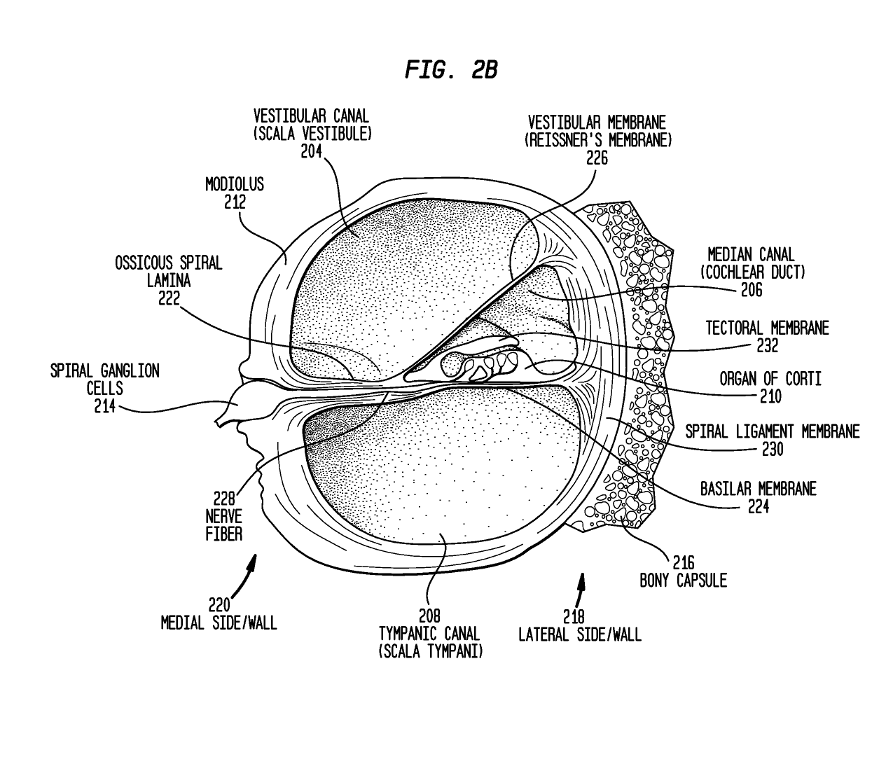

Relevant aspects of a human cochlea are described next below with reference to FIGS. 2A and 2B. FIG. 2A is a perspective view of a human cochlea partially cut-away to display the canals and nerve fibers of the cochlea. FIG. 2B is a cross-sectional view of one turn of the canals of the cochlea illustrated in FIG. 2A. To facilitate understanding, the following description will reference the cochlea illustrated in FIGS. 2A and 2B as cochlea 140, introduced above with reference to FIG. 1.

Referring to FIG. 2A, cochlea 140 is a conical spiral structure comprising three parallel fluid-filled canals, one or more of which are sometimes referred to as ducts. The canals, collectively and generally referred to herein as canals 202, comprise the tympanic canal 208, also know as the scala tympani 208, the vestibular canal 204, also referred to as the scala vestibule 204, and the median canal 206, also referred to as the cochlear duct 206. Cochlea 140 consists of a conical shaped central axis, the modiolus 212, that forms the inner wall of scala vestibule 204 and scala tympani 208. Tympanic and vestibular canals 208, 204 transmit pressure, while medial canal 206 contains the organ of Corti 210 which detects pressure impulses and responds with electrical impulses which travel along the auditory nerve fibers 114 to the brain (not shown).

Referring now to FIG. 2B, separating canals 202 of cochlear 140 are various membranes and other tissue. The Ossicous spiral lamina 222 projects from modiolus 212 to separate scala vestibuli 204 from scala tympani 208. Toward lateral side 218 of scala tympani 208, a basilar membrane 224 separates scala tympani 208 from cochlear duct 206. Similarly, toward lateral side 218 of scala vestibuli 204, a vestibular membrane 226, also referred to as the Reissner's membrane 226, separates scala vestibuli 204 from cochlear duct 206.

The fluid in tympanic and vestibular canals 208, 204, referred to as perilymph, has different properties than that of the fluid which fills cochlear duct 206 and surrounds organ of Corti 210, referred to as endolymph. Sound entering auricle 110 causes pressure changes in cochlea 140 to travel through the fluid-filled tympanic and vestibular canals 208, 204. As noted, organ of Corti 210 is situated on basilar membrane 224 in cochlear duct 206. It contains rows of 16,000-20,000 hair cells (not shown) which protrude from its surface. Above them is the tectoral membrane 232 which moves in response to pressure variations in the fluid-filled tympanic and vestibular canals 208, 204. Small relative movements of the layers of membrane 232 are sufficient to cause the hair cells to send a voltage pulse or action potential down the associated nerve fiber 228. Nerve fibers 228, embedded within spiral lamina 222, connect the hair cells with the spiral ganglion cells 214 which form auditory nerve fibers 114. These impulses travel to the auditory areas of the brain for processing.

The place along basilar membrane 224 where maximum excitation of the hair cells occurs determines the perception of pitch and loudness according to the place theory. Due to this anatomical arrangement, cochlea 140 has characteristically been referred to as being “tonotopically mapped.” This property of cochlea 140 has traditionally been exploited by longitudinally positioning electrodes 148 along carrier member 118 to deliver to a selected region within scala tympani 208 a stimulating signal within a predetermined frequency range.

Portions of cochlea 140 are encased in a bony capsule 216. Referring to FIG. 2B, cochlear bony capsule 216 resides on lateral side 218 (the right side as drawn in FIG. 2B), of cochlea 140. Spiral ganglion cells 214 reside on the opposing medial side 220 (the left side as drawn in FIG. 2B) of cochlea 140. A spiral ligament membrane 230 is located between lateral side 218 of spiral tympani 208 and bony capsule 216, and between lateral side 218 of cochlear duct 206 and bony capsule 216. Spiral ligament 230 also typically extends around at least a portion of lateral side 218 of scala vestibuli 204.

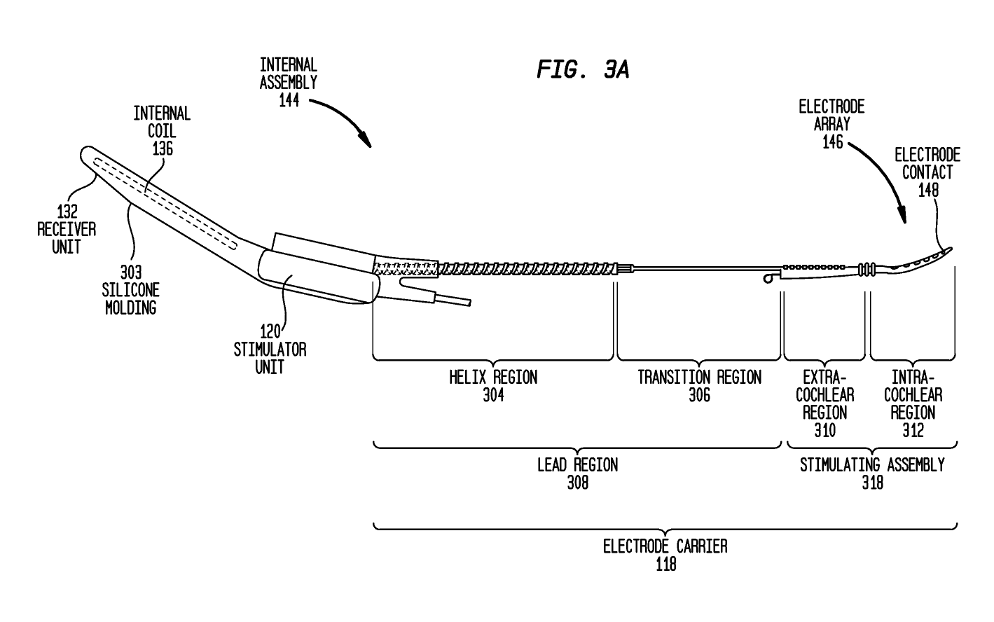

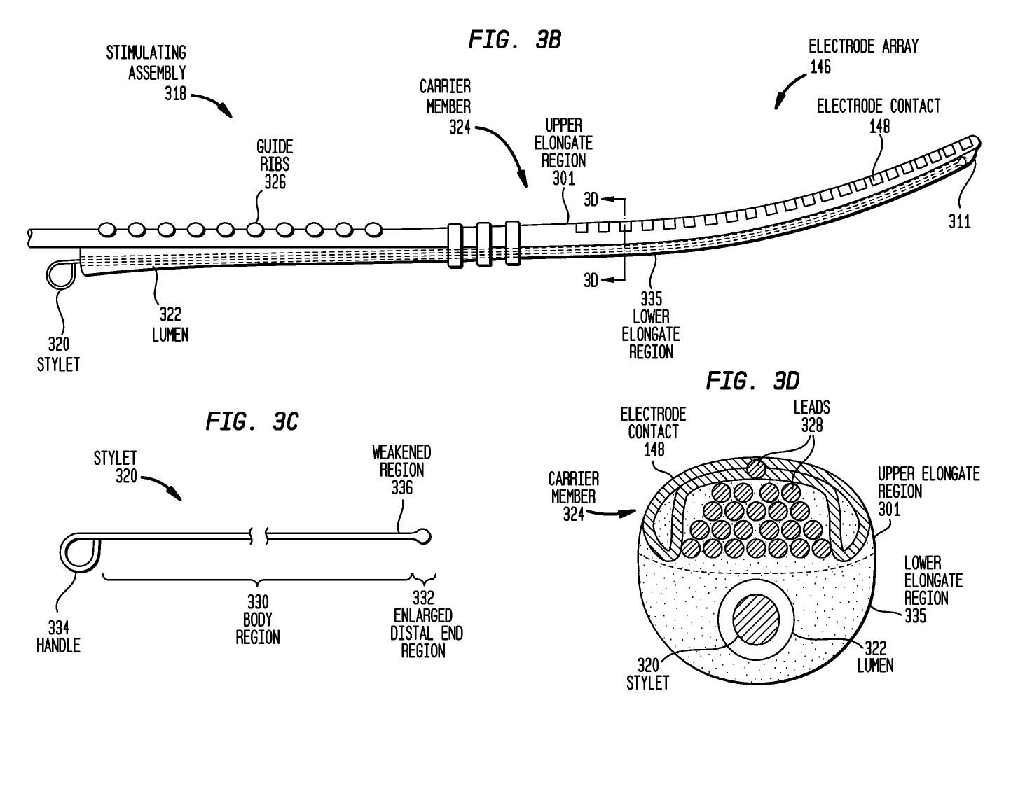

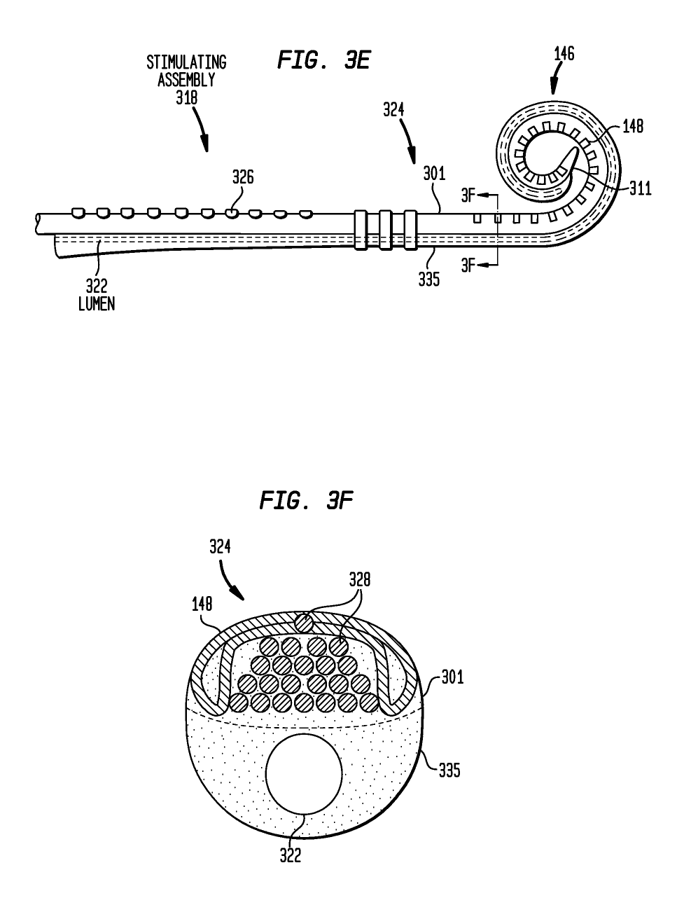

FIG. 3A is a side view of an embodiment of internal assembly 144, introduced above with reference to FIG. 1. FIG. 3B is a side view of stimulating assembly 318 of FIG. 3A, shown in a substantially straight configuration due to the presence of a stylet 320 in stimulating assembly lumen 322.

Internal assembly 144 comprises an internal receiver unit 132, a stimulator unit 120, and an elongate electrode carrier 118. In use, implantable receiver unit 132 may be positioned in a recess of the temporal bone adjacent ear 110 (FIG. 1) of the recipient. Internal receiver unit 132 comprises an internal transcutaneous transfer coil 136. In one embodiment, external coil 130 (FIG. 1) transmits electrical signals to internal coil 136 via a radio frequency (RF) link. Internal coil 136 is typically a wire antenna coil. The electrical insulation of internal coil 136 is provided by a flexible silicone molding 303. Typically, internal coil 136 receives power and data from external coil 130 (FIG. 1), as noted above.

Electrode carrier 118 is comprised of a stimulating assembly 318 configured to be implanted such that a portion of the stimulating assembly referred to as intra-cochlear region 312 is positioned in cochlea 140 (FIG. 1) via, for example, cochleostomy 122 (FIG. 1). Electrode carrier 118 also comprises a cable or lead region 308 which extends from stimulator unit 120 to stimulating assembly 318 to physically and electrically connect stimulating assembly 318 to stimulator unit 120. As such, a proximal end of lead region 308 is connected to stimulator unit 120 while a distal end of the lead region is connected to stimulating assembly 318.

As noted, stimulating assembly 318 comprises an intra-cochlear region 312 which, when implanted, is positioned in cochlea 140 (FIG. 1). Stimulating assembly 318 further comprises an extra-cochlear region 310 which, when implanted, is positioned external to cochlea 140. Intra-cochlear region 312 has an array 146 of electrode contacts 148 configured to deliver stimulation to cochlea 140. Signals generated by stimulator unit 120 are applied by electrode contacts 148 to auditory nerve fibers 114 in cochlear 140, thereby stimulating auditory nerve 114 (FIG. 1).

Lead region 308 is comprised of a helix region 304 connected to stimulator unit 120, and a transition region 306 connecting helix region 304 with stimulating assembly 318. Helix region 304 provides protection against tensile stresses applied to electrode carrier 118. Lead region 308 has a sufficient length to facilitate the implantation of stimulating assembly 318 in a variety of recipients.

FIG. 3B is a side view of stimulating assembly 318 in its straight configuration. A cross-sectional view of the stimulating assembly illustrated in FIG. 3B is shown in FIG. 3D. FIGS. 3E and 3F are side and cross-sectional views, respectively, of the stimulating assembly illustrated in FIGS. 3A and 3B. In FIGS. 3E and 3F stimulating assembly 318 is shown in its pre-curved configuration due to the absence of stylet 320 in lumen 322 of the stimulating assembly. A side view of the stylet is illustrated in FIG. 3C.

Stimulating assembly 318 is comprised of a carrier member 324 in which an array 146 of electrode contacts 148 is disposed in or on (collectively “in” herein) carrier member 324. Carrier member 324 has an upper elongate region 301 in which electrode contacts 146 are positioned, and a lower elongate region 335 in which a lumen 322 is formed.

It has been found that the magnitude of the currents flowing from electrode contacts 148, and the intensity of the corresponding electric fields, is a function of the distance between electrode contacts 148 and modiolus 212. When this distance is relatively great, the threshold current magnitude must be larger than when this distance is relatively small. Moreover, the current from each electrode contact 148 may flow in a number of directions, and the electrical fields corresponding to adjacent electrode contacts may overlap, thereby causing cross-electrode contact interference. To reduce such adverse effects, it is advisable to maintain a minimal distance between carrier member 324 and modiolus 212.

It is also desirable that carrier member 324 be shaped such that the insertion process causes minimal trauma to the sensitive structures of cochlea 140. To position electrode contacts 148 adjacent modiolus 212 (FIGS. 2A, 2B), and to reduce the likelihood of trauma during implantation, carrier member 324 is manufactured to be pre-curved, as shown in FIG. 3E. Specifically, carrier member 324 is manufactured to have a spiral configuration; that is, one or more concentric circles that approximate the curvature of cochlea 140.

As such, carrier member 324 generally adopts a curled or spiral configuration subsequent to implantation into cochlea 140. In the embodiment shown in FIG. 3E, intra-cochlear region 312 of stimulating assembly 318 has a relatively large radius of curvature toward its proximal end and a relatively smaller radius of curvature toward its distal end 311. This increases the effectiveness of the delivery of stimulation to auditory nerve 114 (FIG. 1) while facilitating the atraumatic implantation of stimulating assembly 118.

Stimulating assembly 318 further comprises lumen 322 longitudinally extending through a substantial length of elongate carrier member 324. Lumen 322 extends through a portion of extra-cochlear region 306 and a portion of intra-cochlear region 312. Lumen 322 is configured to receive stylet 320, schematically illustrated in FIG. 3C. It should be appreciated that although embodiments of the present invention are described herein with reference to a stimulating assembly having a continuous lumen as shown in FIGS. 3B and 3E, embodiments of the present invention include stimulating assemblies having a discontinuous lumen as well. Examples of such a stimulating assembly is described in commonly-owned U.S. Pat. No. 7,555,352 and U.S. patent application Ser. No. 12/494,852, both of which are hereby incorporated by reference herein.

Prior to implanting stimulating assembly 318, stylet 320 is inserted into lumen 322 to cause the stimulating assembly to transition from its pre-curved configuration (FIG. 3E) to a substantially straight configuration (FIG. 3A). Thus, in intra-cochlear region 312, stylet 320 performs a straightening function that holds stimulating assembly 318 substantially straight for insertion.

Elongate stimulating assembly 318, while mounted on stylet 320, is inserted through a cochleostomy 122 (FIG. 1) or other natural or man-made aperture until distal end 311 is positioned just short of the basal turn of cochlea 140. Once distal end 311 reaches this approximate location, stimulating assembly 318 is biased forward, or advanced, off stylet 320, causing it to advance further into scala tympani 208 (FIGS. 2A, 2B). As stimulating assembly 318 is biased forward, stylet 320 is withdrawn from carrier member 324, thereby allowing the carrier member to return to its pre-curved configuration. As a result, stimulating assembly 318 continues to adopt a spiral configuration, following the curvature of cochlea 140. This insertion process is sometimes referred to as the Advance Off-Stylet (AOS) mode of implantation. Implantation of a stimulating assembly into a recipient may be difficult at times depending on a variety of factors including, but not limited to, the delicate structure of cochlea 140 and anatomical variations among recipients. For example, in the exemplary application described above, implantation of pre-curved carrier member 324 of stimulating assembly 318 into the delicate structure of cochlea 140 requires a certain degree of skill and patience.

Unfortunately, at times the stimulating assembly is inadvertently advanced too far off the stylet for the location in cochlea 140. This may cause or allow the distal end of the stimulating assembly to fold over on itself as it returns to its pre-curved configuration, or may cause the distal end of the stimulating assembly to perforate the lumen wall. In these and other such circumstances, the delicate structures of the cochlea may be damaged.

Thus, the initial insertion attempt occasionally results in the partial or complete withdrawal of the stylet when the stimulating assembly in a sub-optimal position with respect to the cochlea, allowing the stimulating assembly to at least partially return to its pre-curved configuration. When this occurs, the stimulating assembly can not be advanced further into the cochlea and is withdrawn. Since the stimulating assembly cannot be implanted in its curved configuration, a subsequent attempt to implant the stimulating assembly requires the stimulating assembly to be straightened.

At times, an attempt is made to re-insert the stylet into the lumen to return the stimulating assembly to is pre-insertion configuration shown in FIG. 3A. Traditionally, re-insertion of a stylet into the carrier member lumen has an associated risk of damaging the carrier member and/or stylet. For example, the tip of the stylet may inadvertently puncture or perforate the lumen wall. This may pose a potential pathway for pathogens including harmful bacteria, to migrate from a location external the cochlea into the cochlea. There is also a risk that the stimulating assembly will be damaged while it is being straightened, reducing the integrity of the implanted stimulating assembly. Unfortunately, due to the dimensions of the stimulating assembly and surgical environment, reinserting the stylet even a short distance may cause undetected damage to the stimulating assembly. As such, re-insertion of the stylet is generally best performed by the device manufacturer and any attempt by one other than the device manufacturer is referred to herein as an improper re-insertion.

There are a variety of approaches to insure the integrity of the stimulating assembly. For example, the stimulating assembly is typically provided by the manufacturer in the straight configuration; that is, with the stylet positioned in the carrier member lumen. There are a number of conventional techniques for addressing an inadvertently-withdrawn stylet. For example, one conventional approach has been to have the manufacturer reinsert the stylet using a variety of specialized tools such as a straightening jig as described in U.S. Pat. No. 6,421,569.

Another conventional approach has been to deliver cochlear implants with redundant components, such as a second, or backup, internal assembly 144 or stimulating assembly 318. Should the stimulating assembly not be fully implanted and the stylet partially or fully withdrawn from the stimulating assembly, then that stimulating assembly is set aside and the backup or redundant component is implanted. Subsequently, the unused component is discarded or returned to the manufacturer for inspection and repair.

FIG. 3C is a schematic side view of a stylet of the present invention. Stylet 320 has an elongate body region 330. A handle 334 on the proximal end facilitates the grasping of stylet 320 such as, for example, when adjusting the position of the stylet in lumen 322. It should be appreciated that any grasping feature which facilitates a person controlling the position of stylet 320 and which is appropriate for the particular surgical environment and procedure may serve as handle 334. Alternatively, handle 334 may be configured to be a point of attachment to a tool or other device.

To discourage surgeons from attempting to reinsert stylet 320 and to reduce the likelihood that the stylet will perforate a wall of the lumen when the stylet is re-inserted into the lumen, embodiments of stylet 320 have an enlarged distal end region 332 with a bombous tip. Distal end region 332 has a cross-sectional diameter that is approximately the same as the diameter of lumen 322. As such, distal end region 332 is dimensioned to distribute manual insertion forces over a sufficient region of the lumen to prevent perforation of the lumen wall.

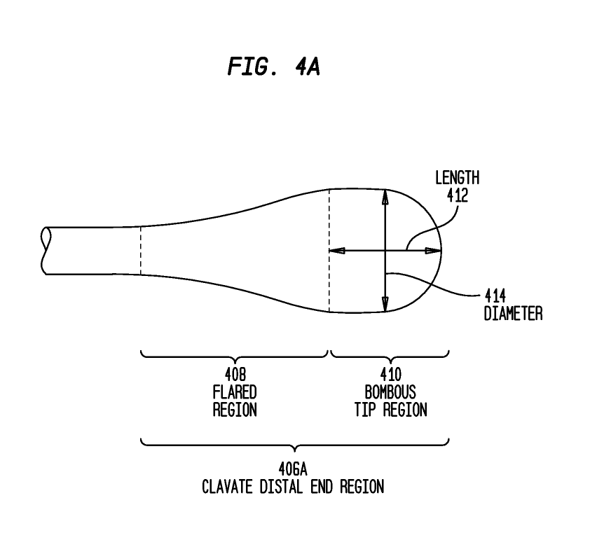

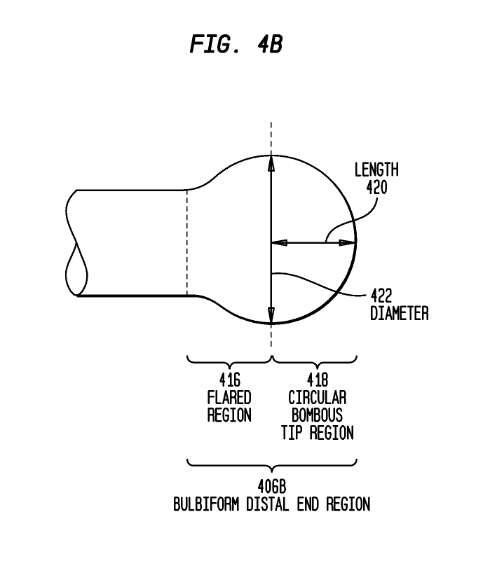

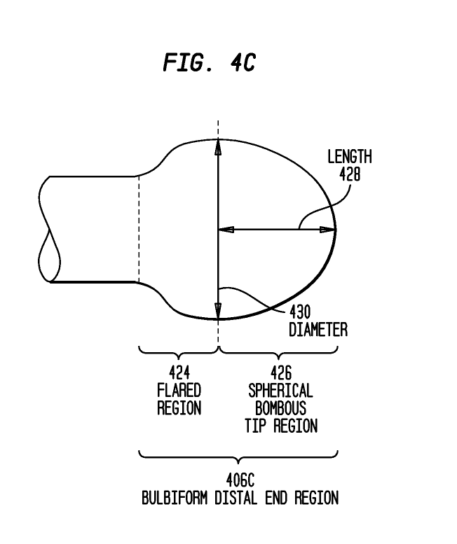

FIGS. 4A-4C are side views of different embodiments of distal end region 332: FIG. 4A is a side view of a clavate distal end region 406A, while FIGS. 4B and 4C are side views of bulbiform distal end regions 406B and 406C, respectively. For ease of description, distal end regions 406 are described herein as having two longitudinally adjacent and contiguous regions: a bombous tip region and a flared region. The bombous tip has a cross-sectional diameter or width that is greater than the cross-sectional diameter of body region 330 and which approximates the diameter of the lumen. It should be understood that in those embodiments in which the tip region has a cross-sectional shape which is other than circular, the cross-sectional diameter of the tip region which approximates the lumen diameter is the largest cross-sectional diameter of the tip region.

The flared region splays out to transition from the smaller diameter body region 330 to the larger diameter bombous tip region. As such, the distal end regions 406 illustrated in FIGS. 4A-4C are depicted with a dashed line at about where the diameter of stylet 320 begins to expand from the smaller diameter body region 330, and a dashed line at about where the diameter of stylet 320 is at a maximum. The two dashed lines, therefore, delineate the flared region. It should be appreciated that such dashed lines are arbitrary and are introduced herein for ease of description.

Returning to FIG. 4A, distal end region 406A comprises a flared region 408 and a bombous tip region 410. As noted, distal end region 406A is clavate. As such, flared region 408 is relatively long, providing a gradual transition from the diameter of body region 330 to the diameter 414 of bombous tip region 410. Bombous tip region 410 is convex and provides a contiguous smooth surface for distal end region 406A.

In FIG. 4B, distal end region 406B has a flared region 416 and a bombous tip 418. In this embodiment, bombous tip 418 is circular, and flared region 416 provides an abrupt transition from the diameter of body region 330 to the diameter 422 of bombous tip 418. As such, distal end region 406B is bulbiform in shape.

In FIG. 4C, bulbiform distal end region 406C has a flared region 424 and a bombous tip 426. In this embodiment, bombous tip 426 is spherical and has a length 428 that is relatively longer than length 420 of bombous tip 418 in FIG. 4B. Flared region 424 is similar to flared region 416 of FIG. 4B, and is shorter in length relative to flared region 408 of distal end region 406A in FIG. 4A.

It should be appreciated that FIGS. 4A-4C depict are a few embodiments of distal end region 332 which may be implemented in a stylet of the present invention. The flared region of such embodiments may have different lengths with outward curvatures which may be gradual, sudden, uneven, and so on. Similarly, the bombous tips of such embodiments may be shaped the same or differently than the bombous tips illustrated in FIGS. 4A-4C.

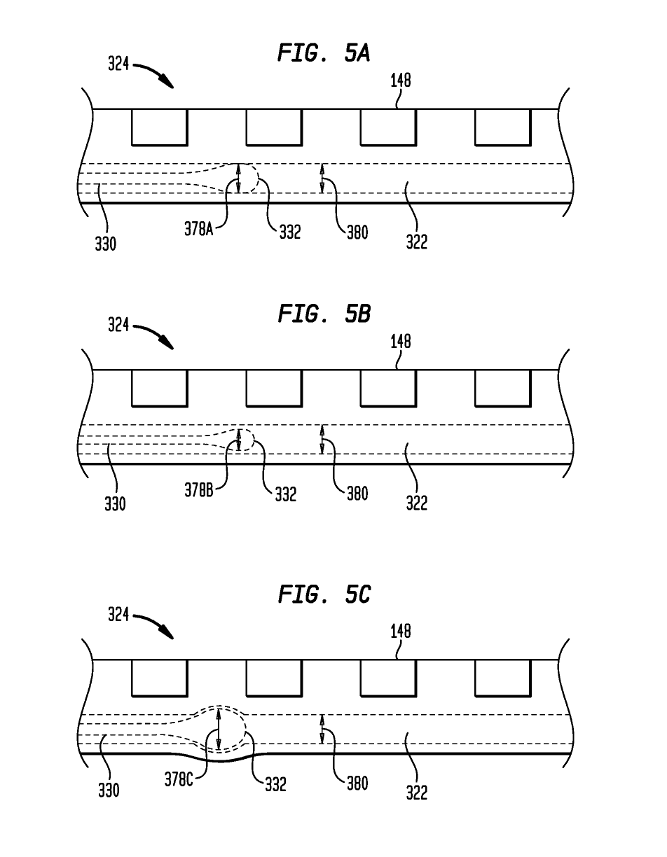

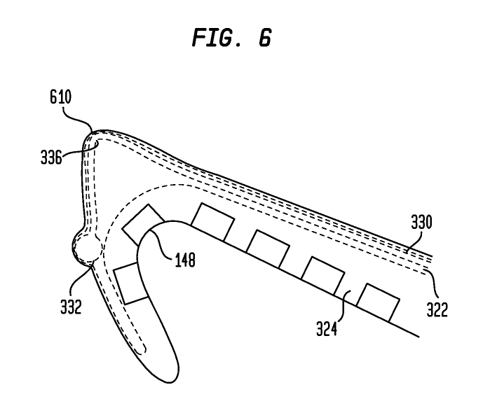

As noted, the width or diameter of distal end region 332 is enlarged; that is, the largest cross-sectional diameter of tip region 410, 418, 426 is approximately the same as the diameter of lumen 322. FIGS. 5A-5C are side views of alternative embodiments of distal end region 332 illustrating different cross-sectional diameters of the bombous tip. In FIG. 5A, the diameter 378A of distal end region 332 is approximately the same as the diameter 380 of lumen 322; in FIG. 5B, the diameter 378B of distal end region 332 is slightly less than diameter 380 of lumen 322; and in FIG. 5C, the diameter 378C of distal end region 332 is slightly larger than diameter 380 of lumen 322. In all three embodiments, the diameter or width 378 of distal end region 332 is approximately the same as the diameter of lumen 322, and in combination with the bombous tip, prevents the stylet from puncturing the wall of lumen 322 when subjected to manual forces applied to the stylet to reinsert the stylet into the carrier member lumen.

Returning to FIG. 3C, in the embodiments illustrated in FIGS. 4A-4C, stylet 320 is, in certain embodiments, a straight platinum wire in which body region 330 has a diameter of approximately 0.125 mm. Distal end 332 has a diameter of approximately 0.18 mm and a smooth radius to stylet body region 330. It should be appreciated that these dimensions are exemplary only. It should also be appreciated that the resultant contact area between the wall of lumen 322 and stylet 320 is approximately 200% of the contact area of conventional stylets.

An optional weakened region 336 may be incorporated into a portion of body region 330 proximate distal end region 332. Weakened region 336 is configured to be relatively less rigid that the other portions of body region 330. As noted, enlarged distal end region 332 is configured to decrease the likelihood that the stylet will perforate lumen 322. Weakened region 336 is configured to bend or buckle in response to relatively high compression forces applied to the stylet thereby supplementing the protection provided by distal end region 332. This is described in greater detail below.

FIG. 6 illustrates how the embodiment of the stylet illustrated in FIG. 3C, stylet 320, interacts with a corresponding electrode carrier member 146 during an unassisted manual insertion of the stylet into the lumen of the carrier member. In this illustrative embodiment, distal end region 332 of stylet 320 has a diameter that is slightly greater than the diameter of lumen 322. In FIG. 6, only the distal-most portion of stimulating assembly 318 is illustrated. As shown in FIG. 3E, this portion of stimulating assembly 318 has a radius of curvature which is relatively smaller that the radius of curvature of the more proximal portions of the stimulating assembly. The radius of curvature is sometimes described as being relatively more gentle (proximally) or aggressive (distally). The bias force of carrier member 146 to return the carrier member to its pre-curved configuration increases distally and decreases proximally due to the relative difference in the radius of curvature along the length of the carrier member.

In the more proximal portions of stimulating assembly 318 not shown in FIG. 6 in which the radius of curvature is relatively gentle, enlarged distal end region 332 and carrier member 324 are configured to encourage stylet 320 to slide through lumen 322 and not perforate lumen 322. That is, the materials and dimensions of carrier member 146 and stylet 320 are such that distal end region 332 does not perforate lumen 322.

However, in the more aggressively curved portion of stimulating assembly 318 shown in FIG. 6, the bias force of carrier member 146 resisting the straightening of the carrier member is relatively greater. As a result, carrier member 146 does not readily uncurl in response to the manual insertion force applied to style 320. This increased resistance increases the axial compressive force applied to stylet 320, resulting in stylet 320 bending or buckling at weakened region 336. In certain embodiments, the compressive force which would result in the buckling of stylet 320 is slightly greater than the compressive force normally applied during a typical reinsertion process. It should be appreciated that the insertion force applied by the surgeon to reinsert stylet 320 may increase in response to this increased resistance. As such, in certain embodiments, stylet 320 has a weakened region 336 that does not buckle until the axial compression force applied to the stylet is some increment greater than the axial compression force normally applied to the stylet during reinsertion, and which is less than the axial compression force which would be applied in response to a manual insertion force that would approximate, but be less than, the manual insertion force that would cause the stylet to perforate the carrier member lumen. Weakened region 336 may be formed, for example, by annealing or thinning the stylet wire in that region.

Further, the materials, manufacturing process and dimensions of carrier member 146 are such that the carrier member deforms in response to the buckling of stylet 320. When stylet 320 buckles, whether at a weakened region 336 or otherwise, the bending of stylet 320 causes carrier member 146 to deform. In the embodiments illustrated in FIG. 6, the resulting deformation region 610 is caused by the buckling of stylet 320 at its weakened region 336. As shown in FIG. 6, this gross deformation causes carrier member 146 to become more curved rather than straighter. This deformation provides visual feedback to the surgeon that the unassisted reinsertion of stylet 320 should be abandoned. Depending on the circumstances, the surgeon may utilize a backup stimulating assembly, as noted above, or use a jig or other appropriate device to reinsert the stylet.

In one embodiment, stylet 320 may be manufactured as follows. First, procure a platinum wire. In one specific embodiment, the platinum wire has a diameter of approximately 0.125 mm, and is 99.95% pure. Cut the wire using, for example, electrode discharge machining (EDM), to create a uniform end with minimal burr. Although there are a variety of well-known techniques which are commonly used, an EDM cutting process has the advantages of being highly automated resulting in greater accuracy and minimal burring.

The cut lengths of the stylet wire are then held in a tooling fixture and loaded onto a CNC-controlled x-y positioning system allowing for automation of enlarged distal end region 332 forming process. An automated laser spot welding process is employed to form the enlarged distal end region 332. Specifically, distal end region 332 is produced on the end of a platinum wire via a pulsed laser. Such technology includes a laser (such as Nd:Yag Neodymium: Yttrium Aluminium Garnet) that utilizes hard-optic or fiber optic beam-delivery. The laser will fire a single beam at the wire generating sufficient heat to melt the end. Due to surface tension, the molten metal forms the enlarged distal end region upon solidification. The process rapidly generates enlarged distal end regions of dimensional and geometrical consistency. As this process is highly automated, uniform enlarged distal end regions with no voids, a smooth surface, and a specified wall thickness, can be consistently formed, and at a faster rate to meet productivity demands. Weakened region 336 may be formed by a variety of techniques such as by thinning, localized annealing, etc.

It will be appreciated by persons skilled in the art that numerous variations and/or modifications may be made to the invention as shown in the specific embodiments without departing from the spirit or scope of the invention as broadly described. For example, in the description of the embodiments described above, the stimulating assembly is implanted by advancing the stimulating assembly off of the stylet. It should be appreciated, however, that the stimulating assembly of the present invention may include stimulating assemblies designed to be inserted using other techniques. More broadly, aspects of the present invention may be implemented in any catheter that is implanted in a recipient using a stylet that is removed from the catheter during implantation. The present embodiments are, therefore, to be considered in all respects as illustrative and not restrictive.

What is claimed is:

1. A cochlear implant comprising:
an elongate implantable stimulating assembly having a carrier member with a lumen longitudinally extending therethrough; and
an elongate stylet configured to be removably inserted into the lumen, the stylet having an elongate body region; and a distal end region comprising a bombous tip having a cross-sectional diameter which is approximately the same as the diameter of the lumen.

2. The cochlear implant of claim 1, wherein the medical device is a stimulating medical device.

3. The cochlear implant of claim 1, wherein the carrier member is an elongate carrier member, and further wherein the lumen longitudinally extends through the elongate carrier member.

4. The cochlear implant of claim 1, wherein the distal end region is bulbiform.

5. The cochlear implant of claim 1, wherein the distal end region is clavate.

6. The cochlear implant of claim 1, wherein the distal end region comprises a flared region and a longitudinally adjacent and contiguous bombous tip region, wherein the bombous tip region has a cross-sectional diameter that approximates the diameter of the lumen, and wherein the flared region splays out to transition from a smaller diameter body region to a larger diameter bombous tip region.

7. The cochlear implant of claim 1, wherein the cross-sectional diameter of the distal end region is not greater than approximately 120% of the lumen diameter.

8. The cochlear implant of claim 1, wherein the carrier member is pre-curved to attain a perimodiolar position in the scala tympani of the cochlea when implanted.

9. The cochlear implant of claim 1, wherein the carrier member is pre-curved to have a radius of curvature that approximates a curvature of a medial side of the scala tympani of the cochlea.

10. The cochlear implant of claim 1, wherein the body region of the stylet comprises a weakened region proximate to the distal end region.

11. An elongate stylet for use with an elongate stimulating assembly of a cochlear implant, the stimulating assembly comprising an implantable carrier member having a lumen extending therethrough and a plurality of electrode contacts disposed on the carrier member, the stylet comprising:
an elongate body region; and
a distal end region comprising a bombous tip having a cross-sectional diameter which is approximately the same as the diameter of the lumen.

12. The stylet of claim 11, wherein the distal end region is bulbiform.

13. The stylet of claim 11, wherein the distal end region is clavate.

14. The stylet of claim 11, wherein the distal end region comprises a flared region and a longitudinally adjacent and contiguous bombous tip region, wherein the bombous tip region has a cross-sectional diameter that approximates the diameter of the lumen, and wherein the flared region splays out to transition from a smaller diameter body region to a larger diameter bombous tip region.

15. The stylet of claim 11, wherein the cross-sectional diameter of the distal end region is not greater than approximately 120% of the lumen diameter.

16. The stylet of claim 11, wherein the carrier member is pre-curved to attain a semi-perimodiolar position in the scala tympani of the cochlea when implanted, and wherein the stylet is sufficiently rigid to retain the carrier member is a substantially straight configuration when the stylet is positioned in the carrier member lumen.

17. The stylet of claim 16, wherein the carrier member is pre-curved to have a radius of curvature that approximates a curvature of a medial side of the scala tympani of the cochlea.

18. The stylet of claim 11, wherein the body region of the stylet comprises a weakened region proximate to the distal end region.

19. The stylet of claim 11, wherein the stylet further comprises:
a proximate handle region to facilitate user control of the configuration, orientation and/or positioning of the stimulating assembly in a recipient.
